(19) United States
(12) Patent Application Publication
(10) Pub. No.: US 2006/0166339 A1
(43) Pub. Date: Jul. 27, 2006

(54) Production of l-aldonolactone

(76) Inventors: Tatsuo Hoshino, Kanagawa-prefecture (JP); Masako Shinjoh, Basel (CH)

Correspondence Address: Stephen M Haracz;Bryan Cave, 1290 Avenue of the Americas, New York, NY, 10104, US

(21) Appl. No.: 10/528,899

(22) PCT Filed: Sep. 18, 2003
(86) PCT No.: PCT/EP03/10366

§ 371 Date: Mar. 23, 2005

(30) Foreign Application Priority Data
Sep. 27, 2002 (EP) 02021596.8

(51) Int. Cl. C12P 17/06; C12P 17/04

(52) U.S. Cl. 435/125; 435/126

(57) ABSTRACT

The present invention provides a process for the production of L-aldonolactone from L-aldohexose, especially for producing L-gulono-1,4-lactone or L-gulonic acid from L-gulose and producing L-galactono-1,4-lactone or L-galactonic acid from L-galactose by a microorganism belonging to the genus Pseudomonas or the genus Gluconobacter.

The present invention is directed to a process for producing L-aldonolactone from L-aldohexose by a microorganism belonging to the genus Pseudomonas or the genus Gluconobacter.

The L-aldonolactones L-gulono-1,4-lactone and L-galactono-1,4-lactone, respectively, are intermediates in the biosynthesis of L-ascorbic acid (vitamin C) by animals and plants. The proposed pathway for the synthesis of vitamin C in animals starts from D-glucose and goes on via the intermediates D-glucose-6-phospate, D-glucose-1-phosphate, UDP-D-glucose, UDP-D-glucuronic acid, D-glucuronic acid, L-gulonic acid, L-gulono-1,4-lactone and 2-keto-L-gulono-1,4-lactone to the endproduct vitamin C. The proposed pathway for the synthesis of vitamin C in plants starts from D-glucose and goes on via the intermediates D-glucose-6-phospate, D-fructose-6-phospate, D-mannose-6-phosphate, GDP-D-mannose, GDP-L-galactose, L-galactose-1-phosphate, L-galactose, L-galactono-1,4-lactone and 2-keto-L-galactono-1,4-lactone to the endproduct vitamin C.

Feasibility studies on the biotechnological synthesis of vitamin C were performed for many years since the “Reichstein method” was established in 1934. The microorganisms Gluconobacter oxydans DSM 4025, Candida albicans and Saccharomyces cerevisiae oxidize L-galactono-1,4-lactone to vitamin C. Saccharomyces cerevisiae and Candida albicans possess D-arabinose dehydrogenase catalyzing the production of D-arabinono-1,4-lactone and L-galactono-1,4-lactone from D-arabinose and L-galactose, respectively. However, there were no reports describing the possibility of biological vitamin C production from another L-hexose as intermediate, that is, L-idose, L-gulose, and L-talose, with a configuration corresponding to that of vitamin C (at positions C4 and C5).

The present invention provides a process for the production of L-aldonolactone from L-aldohexose by a microorganism capable of producing L-aldonolactone from L-aldohexose, and, optionally, isolating the L-aldonolactone from the reaction mixture.

The L-aldonolactones produced by the process of the present invention are selected from the group consisting of L-gulono-1,4-lactone, L-gulonic acid, L-galactono-1,4-lactone, and L-galactonic acid.

As used herein, “L-gulono-1,4-lactone (and its acid form, L-gulonic acid)” or “L-galactono-1,4-lactone (and its acid form, L-galactonic acid)” means co-existing mixture of the lactone form together with the acid form as the result of physicochemical equilibrium.

The L-aldohexoses used in the process of the present invention for the production of L-aldonolactones are selected from L-gulose or L-galactose.

Thus, in the present invention L-gulono-1,4-lactone and its acid form, L-gulonic acid, is produced from L-gulose and L-galactono-1,4-lactone and its acid form, L-galactonic acid, is produced from L-galactose.

L-Aldohexoses like L-gulose, L-galactose, L-idose, and L-talose are rare sugars, which are basically produced by chemical methods and are commercially high-cost compounds. However, biological preparations of L-gulose and L-galactose have been recently reported. L-Gulose production from D-sorbitol by enzyme A of G. oxydans DSM 4025 was reported in EP 0 832 974 A2. L-Gulose production from L-sorbose by L-ribose isomerase was disclosed in U.S. Pat. No. 6,037,153. L-galactose production from L-sorbose is reported by Izumori et al. (2001 Annual Meeting of the Society for Bioscience and Bioengineering, Japan).

Microorganisms capable of producing L-aldonolactone from L-aldohexose as of the present invention may be selected from Pseudomonas or Gluconobacter. A preferred microorganism is Pseudomonas putida or Gluconobacter oxydans. More preferred are P. putida ATCC 21812 or G. oxydans IFO 3293. The microorganism may also be a biologically and/or taxonomically homogeneous culture of a microorganism having the identifying characteristics of P. putida ATCC 21812 or G. oxydans IFO 3293.

The strain P. putida ATCC 21812 is available from the American Type Culture Collection (12301 Parklawn Drive, Rockville, Md. 20852, USA). Strain G. oxydans IFO 3293 is available from the Institute for Fermentation, Osaka (17-85, Juso-honmachi 2-chome, Yodogawa-ku, Osaka 532, Japan).

As used herein, the term “biologically and/or taxonomically homogeneous culture” includes, besides P. putida ATCC 21812 or G. oxydans IFO 3293, also a microorganism of a different species/genus but which has the identifying characteristics of P. putida ATCC 21812 or G. oxydans IFO 3293. The decision whether a microorganism belongs to such homogeneous culture should be based on 16S rRNA sequence comparison.

The microorganism “Pseudonmonas putida” and “Gluconobacter oxydans” also include synonyms or basonyms of such species having the same physico-chemical properties, as defined by the International Code of Nomenclature of Prokaryotes.

The present invention, therefore, provides a process for the production of L-aldonolactone from L-aldohexose, especially for producing L-gulono-1,4-lactone or L-gulonic acid from L-gulose, or L-galactono-1,4-lactone or L-galactonic acid from L-10 galactose by a microorganism belonging to the genera Pseudomonas or Gluconobacter capable of producing L-aldonolactone from L-aldohexose, and isolating the L-aldonolactone from the reaction mixture. The process may be conducted in a growing culture or a resting cell reaction.

Thus, it is an embodiment of the present invention to provide a process for the production of L-aldonolactone from L-aldohexose as above, wherein a microorganism capable of producing L-aldonolactone from L-aldohexose as defined above is used in a growing culture or a resting cell reaction.

In the present invention, mutants of the above mentioned strains can also be used. As used herein the term “mutation” refers to an alteration in the genomic sequence of the microorganism, which may be introduced by any convenient means including, for example, chemical and UV mutagenesis, followed by screening or selection for a desired phenotype, construction of dysfunctional genes in vitro by recombinant techniques used to replace the intact counterparts of the genes in the genome of the microorganism, by single and double cross-over recombinations, and other well known techniques. See, Sambrook, et al., Molecular Cloning, A Laboratory Manual, 2nd Ed., Cold Spring Harbor Laboratory Press (1989) and, Harwood and Cutting, Molecular Biology Methods For Bacillis, John Wiley and Sons (1990), pp. 27-74. Suitable mutagens include, but are not limited to, ultraviolet-ray, X-ray, γ-ray and nitrous acid. Furthermore, a mutant strain can be obtained by isolating a clone occurring by spontaneous mutation thereof in any of the ways per se well known for the purpose by one skilled in the art.

The microorganisms may be cultured in an aqueous medium supplemented with appropriate nutrients under aerobic conditions. The cultivation may be conducted at a pH between about 1.0 and 9.0, preferably between about 2.0 and 8.0. While the cultivation period varies depending on pH, temperature and nutrient medium used, usually 1 to 120 hours will bring about favorable results. A preferred temperature range for carrying out the cultivation is from about 13° C. to 45° C., more preferably from about 18° C. to 42° C.

Thus, it is an object of this invention to provide a process for the production of L-aldonolactone from L-aldohexose by a microorganism capable of producing L-aldonolactone from L-aldohexose, wherein the process is conducted for 1 to 120 h at a pH range of from about 1 to about 9 and at a temperature in the range of from about 13° C. to about 45° C. In a preferred embodiment, the process as above is conducted at a pH range of from about 2 to about 8 and at a temperature in the range of from about 18° C. to about 42° C.

The concentration of L-aldohexose in a reaction mixture can vary depending on other reaction conditions, but, in general, is between 1 g/l and 300 g/l, preferably between 10 g/l and 200 g/l.

It is usually required that the culture medium contains such nutrients as assimilable carbon sources, digestible nitrogen sources and inorganic substances, vitamins, trace elements and other growth promoting factors. Examples of assimilable carbon sources include, but are not limited to, glycerol, D-glucose, D-mannitol, D-fructose, D-arabitol, D-sorbitol and L-sorbose.

Various organic or inorganic substances may also be used as nitrogen sources, such as yeast extract, meat extract, peptone, casein, corn steep liquor, urea, amino acids, nitrates, ammonium salts and the like. As inorganic substances, magnesium sulfate, potassium phosphate, ferrous and ferric chlorides, calcium carbonate and the like may be used.

Vitamins such as biotin, cyanocobalamin, thiamin•HCl, pyridoxine•HCl, Ca-pantothenate, folic acid, inositol, niacin, p-aminobenzoic acid, and riboflavin are useful for the present invention.

Suitable trace elements as used for the present invention are selected from rare metals, such as Mo, Mn, Cu, Co, and Zn in the form of inorganic salts, e.g., Na₂MoO₄.2H₂O, vitamins, amino acids, purines, and pyrimidines. Other growth promoting factors include, but are not limited to, amino acids such as tryptophan or histidine, purines such as adenine or guanine, and pyrimidines such as cytosine and thymine.

After the reaction, L-aldonolactone may be recovered from the reaction mixture by the combination of various kinds of chromatography, for example, thin layer chromatography, adsorption chromatography, ion-exchange chromatography, gel filtration chromatography or high performance liquid chromatography. The reaction product can also be used as a substrate for a further reaction as it is in the reaction mixture of this invention without purification.

The following examples are provided to further illustrate the process of the present invention. These examples are illustrative only and are not intended to limit the scope of the invention in any way.

### EXAMPLE 1

### Production of L-gulono-1,4-lactone from L-gulose by P. putida or G. oxydans

P. putida ATCC 21812 and G. oxydans IFO 3293 were grown on MB agar medium consisting of 2.5% mannitol, 0.5% yeast extract (Difco), and 0.3% Bactopeptone (Difco) at 30° C. for 48 h. The resulting cells were used for a resting cell reaction. The reaction mixture (1 ml) consisting of 2% L-gulose, 0.3% NaCl, 1% CaCO₃ and 1 mM phenazine methosulfate was incubated at room temperature for 17 h. The produced amounts of L-gulono-1,4-lactone and L-gulonic acid were assayed by thin layer chromatography (TLC) and high performance liquid chromatography (HPLC) as summarized in Table 1. The TLC assay was performed with silica gel (Kiesel gel 60F₂₅₄, 0.25 mm, Merck), the solvent system consisting of n-propanol-H₂O-1% H₃PO₄—HCOOH (400:100:10:1). The HPLC assay was performed at 210 nm with a YMC-Pack Polyamine II column (150×4.6 mm I.D.; YMC CO., Ltd., Kyoto, Japan) and with acetonitrile-50 mM NH₄H₂PO₄ (67:33). The TLC plate was sprayed with 0.5% KIO₄ solution and then sprayed with the mixture of an equal volume of tetrabase-saturated 2N CH₃COOH and 15% MnSO₄ solution. The products L-gulono-1,4-lactone and L-gulonic acid were detected as white spots.

TABLE 1
Tube resting reaction with L-gulose as a substrate

| | TLC | | HPLC (mM) |
|---|---|---|---|
| Strain | L-GuL | L-GuA | L-GuL + L-GuA |
| Pseudomonas putida ATCC 21812 | nd | ++ | 16.5 |
| Gluconobacter oxydans IFO 3293 | nd | + | 5.2 |
| No cells | nd | nd | nd |

L-GuL: L-gulono-1,4-lactone;
L-GuA: L-gulonic acid;
++: more than 5 mM;
+: 5 mM or less;
nd: not detectable

The reactions with L-gulose as the substrate were also done in a mini-resting-cell reaction with 100 μl reaction mixture consisting of 2% L-gulose, 0.3% NaCl, 1% CaCO₃. Escherichia coli HB101 grown on Luria Bertani (LB) agar at 37° C. for 1 day was also used in this reaction. Amounts of produced L-gulono-1,4-lactone and L-gulonic acid are shown in Table 2.

TABLE 2
Mini-resting reaction with L-gulose as a substrate

TLC

| Strain | L-GuL | L-GuA |
|---|---|---|
| Pseudomonas putida ATCC 21812 | nd | + |
| Gluconobacter oxydans IFO 3293 | + | + |
| Escherichia coli HB101 | nd | nd |
| No cells | nd | nd |

L-GuL: L-gulono-1,4-lactone;
L-GuA: L-gulonic acid;
+: 5 mM or less;
nd: not detectable

### EXAMPLE 2

### Production of L-galactono-1,4-lactone from L-galactose

P. putida ATCC 21812 and G. oxydans IFO 3293 were grown on MB agar plate at 30° C. for 48 h. Saccharomyces cerevisiae ATCC 9763 was grown on the YN medium (Difco) with 2% D-glucose and 1.8% agar at 30° C. for 48 h. E. coli HB101 grown on Luria Bertani (LB) agar at 37° C. for 1 day was also used in this reaction. The resulting cells were used for a resting cell reaction. The reaction mixture (100 μl) consisted of 2% L-galactose, 0.3% NaCl, 1% CaCO₃ and the cells (OD600≈20) was incubated at room temperature for 23 h. The produced amounts of L-galactono-1,4-lactone and L-galactonic acid were assayed by TLC and HPLC as summarized in Table 3. P. putida ATCC 21812 and G. oxydans IFO 3293 produced significantly more L-galactono-1,4-lactone together with L-galactonic acid than S. cerevisiae ATCC 9763 and E. coli HB101, both of which produced undetectable amounts of L-galactono-1,4-lactone and L-galactonic acid.

TABLE 3
Mini-resting reaction with L-galactose as a substrate

| | TLC | | HPLC (mM) |
|---|---|---|---|
| Strain | L-GaL | L-GaA | L-GaL + L-GaA |
| Pseudomonas putida ATCC 21812 | ++ | ++ | 75.8 |
| Gluconobacter oxydans IFO 3293 | + | + | 10.0 |
| Saccharomyces cerevisiae | nd | nd | nd |
| ATCC 9763 | | | |
| Escherichia coli HB1O1 | nd | nd | nd |
| No cells | nd | nd | nd |

L-GaL: L-galactono-1,4-lactone;
L-GaA: L-galactonic acid;
++: more than 10 mM;
+: 10 mM or less;
nd: not detectable

1. A process for the production of L-aldonolactone from L-aldohexose by a microorganism capable of producing L-aldonolactone from L-aldohexose, and, optionally, isolating the L-aldonolactone from the reaction mixture.

2. The process according to claim 1 wherein the L-aldonolactone is selected from the group consisting of L-gulono-1,4-lactone, L-gulonic acid, L-galactono-1,4-lactone, and L-galactonic acid.

3. The process according to claim 1 wherein the L-aldohexose is selected from L-gulose or L-galactose.

4. The process according to claim 1 wherein the microorganism is selected from Pseudomonas or Gluconobacter.

5. The process according to claim 4 wherein the microorganism is Pseudomonas putida or Gluconobacter oxydans.

6. The process according to claim 4 wherein the microorganism is P. putida ATCC 21812 or G. oxydans IFO 3293.

7. The process of claim 1 wherein the microorganism is used in a growing culture or a resting cell reaction.

8. The process of claim 1 wherein the process is conducted for 1 to 120 h at a pH in the range of from about 1 to about 9 and at a temperature in the range of from about 13° C. to about 45° C.

9. The process of claim 8 wherein the process is conducted for 1 to 120 h at a pH in the range of from about 2 to about 8 and at a temperature in the range of from about 18° C. to about 42° C.
